(19) United States
(12) Patent Application Publication
(10) Pub. No.: US 2022/0268774 A1
(43) Pub. Date: Aug. 25, 2022

(54) METHOD OF ELEVATING PREDICTION ACCURACY OF GROUPING SUBJECTS WITH SEVERE DENGUE INFECTION

(71) Applicant: NATIONAL CHENG KUNG UNIVERSITY, Tainan City (TW)

(72) Inventors: Tzong-Shiann HO, Tainan City (TW); Ya-Lan LIN, Tainan City (TW); Hong-Jyun HUANG, Tainan City (TW); Yung-Chun CHUANG, Tainan City (TW); Yu-Wei CHENG, Tainan City (TW)

(21) Appl. No.: 17/533,535

(22) Filed: Nov. 23, 2021

(30) Foreign Application Priority Data
Feb. 23, 2021 (TW) 110106367

(51) Int. Cl. G01N 33/577; G01N 33/68

(52) U.S. Cl. CPC G01N 33/577; G01N 33/6854; G01N 33/6893; G01N 2800/26

(57) ABSTRACT

The present invention relates to a method of elevating prediction accuracy of grouping subjects with severe dengue infection. In the method, a non-structural protein 1 (NS1) and an endogenous anti-NS1 antibody of dengue virus in an ex vivo biological specimen are detected and crossly compared, leading in reduce of false negative rates of testing results, as well as elevating grouping accuracy of patients with severe dengue infection.

### RELATED APPLICATIONS

This application claims priority to Taiwan Application Serial Number 110106367, filed Feb. 23, 2021, which is herein incorporated by reference.

### BACKGROUND

### Field of Invention

The present invention relates to a medical examination method. More specifically, the present invention relates to a method of elevating prediction accuracy of grouping subjects with severe dengue infection by detecting a non-structural protein 1 (NS1) and an endogenous anti-NS1 antibody of dengue virus in an ex vivo biological specimen.

### Description of Related Art

Dengue fever is a disease that quickly spreads and has a short course, and about 390 million people are infected by dengue virus worldwide every year. In the past 20 years, Taiwan has experienced several regional dengue fever epidemics. In addition to the epidemic in Southern Taiwan, there have been clustered outbreaks of indigenous dengue cases in New Taipei City and Taichung City that are not the main affected areas, which shows that dengue fever has a trend of localization, and its threat spreads to the whole Taiwan. Dengue fever has become an important emerging infection and public health problem in Taiwan.

The clinical symptoms of dengue patients vary greatly, from fever like a common cold to fatal dengue shock syndrome or dengue hemorrhagic fever. Early diagnosis of severe dengue fever can provide timely disease monitoring and management. However, current test techniques yet cannot meet the clinical need to predict the severity of dengue fever. New research in recent years has further found that NS1 is an important viral toxin, which is known to cause important pathogenic effects such as plasma leakage, dysfunction of blood coagulation, and thrombocytopenia during severe dengue infection. In addition, an anti-NS1 antibody has been confirmed in animal experiments to have the effect of treating hemorrhagic lesions caused by dengue infection.

It has been found in previous studies that, the viral toxin NS1 forms a complex with thrombin or prothrombin in serum samples from patients with dengue infection, prolonging activation of partial thromboplastin and thus causing severe bleeding. Therefore, the current test process involves first collection and testing (briefly referred to as first collection) for suspected dengue infection cases and fast screening for NS1 antigen of the dengue virus. If the fast screening result in the first collection is negative, the process further involves second collection and testing (briefly referred to as second collection) and dengue virus-specific real-time PCR, RT-PCR, and IgM/IgG tests, where the IgM/IgG test refers to seroconversion of anti-dengue IgM or IgG antibodies or at least four-fold increase of a positive result of IgG antibodies in the serum of the second collection.

However, the current test process has a high false negative rate of testing results of patients with mild dengue fever, easily causing misjudgment. In view of this, it is in urgent need to provide a method of elevating prediction accuracy of grouping subjects with severe dengue infection, so as to solve the conventional problem of a high false negative rate of testing results of dengue fever patients.

### SUMMARY

Accordingly, an aspect of the present invention provides a method of elevating prediction accuracy of grouping subjects with severe dengue infection, in which a non-structural protein 1 (NS1) and an endogenous anti-NS1 antibody of dengue virus in an ex vivo biological specimen are detected and crossly compared, leading in reduction of false negative rates of testing results, as well as elevating prediction accuracy of grouping patients with severe dengue infection.

According to the foregoing aspect of the present invention, a method of elevating prediction accuracy of grouping subjects with severe dengue infection is provided. In an example, the grouping method includes: providing an ex vivo biological specimen; performing at least one detection step on the ex vivo biological specimen, so as to obtain a first test result and a second test result; and crossly comparing the first test result and the second test result so as to obtain a grouping result.

In the aforementioned embodiment, the ex vivo biological specimen has not been diagnosed or differentially diagnosed with a dengue virus infection or a suspected dengue virus infection.

In the aforementioned embodiment, the first test result is corresponding to NS1 and/or an NS1 complex of dengue virus, and the second test result is corresponding to an endogenous anti-NS1 antibody.

In the aforementioned embodiment, when at least one of the first test result and the second test result is positive, a subject corresponding to the ex vivo biological specimen can be classified as a severe dengue infection group.

In the aforementioned embodiment, the ex vivo biological specimen includes blood, urine, saliva, tissue fluid and/or lymphatic fluid.

In the aforementioned embodiment, the first test result is obtained by detecting the NS1 and/or NS1 complex with an antibody, the antibody is an endogenous anti-NS1 antibody, and the NS1 complex includes NS1-thrombin or NS1-prothrombin.

In the aforementioned embodiment, the serotypes of the dengue virus include type 1, type 2, type 3 and type 4.

In the aforementioned embodiment, the endogenous anti-NS1 antibody is a humanized antibody (hAb), and the endogenous anti-NS1 antibody includes a first antibody and a second antibody, where the first antibody may, for example, specifically recognize the 109^{th} to 122^{nd} amino acid residues of the NS1 and the second antibody may, for example, specifically recognize the 114^{th} to 119^{th} amino acid residues of the NS1. In an instance, an isotype of the endogenous anti-NS1 antibody is IgG and/or IgM, and the second test result is a content ratio of the first antibody to the second antibody.

In the aforementioned embodiment, the subject can be a mammal, for example, a human being.

In the aforementioned embodiment, when both of the first test result and the second test result are negative, the subject corresponding to the ex vivo biological specimen can be classified as a non-severe dengue infection group.

In the aforementioned embodiment, the at least one detection step includes an enzyme-linked immunosorbent assay (ELISA), western blot analysis, lateral laminar flow immunoassay, multiple immunoassay, radio immunoassay, immunoradiometric analysis, fluorescence immunoassay, chemiluminescence immunoassay and/or immunoturbidimetry.

### BRIEF DESCRIPTION OF THE DRAWINGS

The disclosure can be more fully understood by reading the following detailed description of the embodiment, with reference made to the accompanying drawings as follows.

FIG. 1 shows a content ratio of modified NS1-WD IgG/NS1 IgG in sera of various dengue fever patients according to an embodiment of the present invention.

FIG. 2 shows a consistency result for specificity and sensitivity of prediction of subjects with severe dengue infection according to a conventional method.

FIG. 3 shows a consistency result for specificity and sensitivity of prediction of subjects with severe dengue infection in terms of NS1 antigens in the sera of dengue fever patients according to an embodiment of the present invention.

FIG. 4 shows a consistency result for specificity and sensitivity of prediction of subjects with severe dengue infection in terms of a proportion of the endogenous anti-NS1 antibody in the sera of dengue fever patients according to an embodiment of the present invention.

FIG. 5 shows a consistency result for specificity and sensitivity of prediction of subjects with severe dengue infection in terms of a proportion of the endogenous anti-NS1 antibody in the sera of dengue fever patients by means of mouse antibody detection according to a comparative example.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

All documents cited herein are deemed to be specifically and individually incorporated into references through citation of each individual document or patent application. Where a definition or use of a term in an incorporated reference is inconsistent or contrary to the definition of that term provided herein, the definition of that term provided herein applies and the definition of that term in the reference does not apply.

To explain the specification, the following definitions are applicable. Unless inappropriate in the context, the terms “(a/an)” and “(the/said)” mentioned herein are defined as “one or more” and include plural forms. Additional definitions are set forth throughout the detailed description.

As described above, the present invention provides a method of elevating prediction accuracy of grouping subjects with severe dengue infection, in which a non-structural protein 1 (NS1) and an endogenous anti-NS1 antibody of dengue virus in an ex vivo biological specimen are detected and crossly compared, leading in reduction of false negative rates of testing results, as well as elevating prediction accuracy of grouping patients with severe dengue infection.

The term “dengue virus” mentioned herein can be used alternately with “dengue fever virus” and “DENV”. The serotypes of the dengue virus can include but be not limited to type 1, type 2, type 3 and type 4.

In serum samples from patients with dengue infection, NS1 of the dengue virus forms a complex with thrombin or prothrombin, prolonging activation of partial thromboplastin and thus causing severe bleeding. In the prior art, it is determined whether a subject is infected with dengue virus by detecting whether there is a complex of NS1 and thrombin or a complex of NS1 and prothrombin in an in vitro biological sample. However, among patients with dengue infection, different patients vary greatly in severity of the disease, and a severe disease can even lead to death. The current test process merely tests the complex of NS1 and thrombin or the complex of NS1 and prothrombin, and the test results have a high false negative rate, thus failing to accurately determining the severity of the disease. As a result, it is likely to neglect the disease condition and delay the treatment.

Therefore, the method of the present invention performs at least one test step for an ex vivo biological specimen, and crossly compares a first test result and a second test result to obtain a grouping result, so as to determine whether the ex vivo biological specimen contains NS1 and an endogenous anti-NS1 antibody of dengue virus, leading in reduction of false negative rates of testing results, as well as elevating prediction accuracy of grouping patients with severe dengue infection. It is noted that, the terms “test” and “detect” (or examine) mentioned herein can be alternately used, and the terms “accuracy” and “accuracy rate” can also be alternately used.

The “ex vivo biological specimen” generally refers to a range of influence within a subject with dengue virus infection and is not particularly limited, which can include but be not limited to blood (e.g., serum, plasma, or whole blood), urine, saliva, lymphatic fluid, or tissue fluid; or nearby tissues or cells through which the blood, urine, lymphatic fluid, or tissue fluid flows. In some embodiments, the ex vivo biological specimen preferably contains cells infected with the dengue virus. The cells can include but be not limited to nerve cells, muscle cells, liver cells, endothelial cells, blood cells, and lymphocytes; and preferably include endothelial cells or blood cells of mammals. In other embodiments, the ex vivo biological specimen can be, for example, a fresh, tissue-cultured, or refrigerated or frozen sample. In some specific examples, the ex vivo biological specimen can be subjected to conventional pretreatment (for example, purification, centrifugation, extraction, or concentration), so as to increase the concentration of a substance (for example, the NS1 and/or NS1 complex, or the endogenous anti-NS1 antibody) to be detected.

The “first test result” is corresponding to the NS1 and/or NS1 complex of the dengue virus. Specifically, the first test result is obtained by detecting the NS1 and/or NS1 complex with an antibody, where the antibody is an exogenous anti-NS1 antibody. In an instance, the NS1 complex includes NS1-thrombin or NS1-prothrombin.

The “exogenous antibody” can be, for example, a monoclonal antibody or a polyclonal antibody. In some embodiments, the antibody that specifically recognizes the NS1 can be, for example, a monoclonal antibody. In other embodiments, the antibody that specifically recognizes the NS1 complex can be, for example, a polyclonal antibody.

The exogenous antibody includes an antibody-based binding moiety, or immunoglobulin molecules and their immunologically active determinants, for example, molecules containing an antigen-binding site that binds immune-specifically to the NS1 or the complex. The type of the exogenous antibody can include but be not limited to IgG, IgA, IgM, IgE, or the like, instead of limitation.

The exogenous antibody can also include an antigen-binding fragment that specifically reacts with the NS1 or the NS1 complex. The antigen-binding fragment is not limited in structure, and in consideration of the structural stability of the complementarity-determining region (CDR), can have a complete antibody structure or simplified antibody structure, such as a single-chain variable fragment (scFv), scFv dimer [(scFv)₂], scFv trimer [(scFv)₃], a variable fragment (Fv), a Fab fragment, a Fab′ fragment, a F(ab′)₂ fragment, a nanobody (also referred to as a single domain antibody (sdAb) or a heavy-chain antibody), or any combination of the above.

After a patient is infected with the dengue virus, in addition to the NS1 and/or the NS1 complex that is detected in the ex vivo biological specimen, an anti-NS1 “endogenous antibody” is also produced in the body. The inventors also find that the concentration of the NS1 and/or NS1 complex and the concentration of the endogenous antibody against a specific NS1 peptide sequence in the body of the patient are relevant to the severity of the dengue disease. In an embodiment, the endogenous anti-NS1 antibody can be detected in the ex vivo biological specimen, so as to obtain a second test result.

In this embodiment, the endogenous anti-NS1 antibody is not limited in category and can include, but is not limited to, a first antibody and a second antibody. In some instances, the first antibody refers to an endogenous antibody that specifically recognizes the 109^{th} to 122^{nd} amino acid residues of the NS1 of the dengue virus, where the 109^{th} to 122^{nd} amino acid residues of the NS1 can be defined as modified NS1-WD peptide, and that is referred hereafter to as an antibody against modified NS1-WD peptide or an anti-NS1-WD peptide antibody. It is found in the past clinical studies that the higher the concentration of such an anti-NS1-WD peptide antibody in the body, the less likely it is for the patient to develop a severe disease. In addition, the quality and quantity of the anti-NS1-WD peptide antibody are also relevant to the severity of the disease.

In some other instances, the second antibody refers to an endogenous antibody that specifically recognizes the 114^{th} to 119^{th} amino acid residues of the NS1 of the dengue virus, where the 114^{th} to 119^{th} amino acid residues of the NS1 belong to a conserved sequence of four serotypes of the dengue virus and facilitate recognition of NS1 of the four serotypes of the dengue virus, and that is also referred to as an antibody against NS1 of all serotypes or an anti-NS1 antibody.

In other embodiments, the isotype of the endogenous anti-NS1 antibody is not limited and can be, for example, IgG and/or IgM. In some specific examples, the second test result refers to a content ratio of the first antibody to the second antibody.

In some specific examples, a humanized antibody (hAb) can be used to specifically recognize the endogenous anti-NS1 antibody. A method for producing the hAb belongs to common knowledge in the art of the present invention. In some embodiments, the skeleton of a recipient human antibody can be used, and a CDR sequence of a rodent antibody is used to replace the corresponding sequence of the human antibody, so as to obtain a hAb, and such a hAb belongs to a human-mouse chimeric antibody. In examples of the human-mouse chimeric antibody, a human antibody germline sequence available from a public database can be selected for the skeleton of the recipient human antibody, where the ethnic group of the skeleton of the recipient human antibody is not particularly limited and depends on the ex vivo biological specimen to be detected.

The “individual”, “subject”, or “patient” mentioned herein refer to a mammal. In a specific example, the individual, subject, or patient can be, for example, a human being.

The dengue fever patients with “mild symptoms” mentioned herein are those who show warning signs (or referred to as warning symptoms) or have no warning signs, where the warning signs can include but be not limited to, for example, abdominal pain or tenderness, persistent vomiting, clinical fluid accumulation, mucosal bleeding, and the like. In other embodiments, mild patients who have no warning signs can be classified as group A patients, while mild patients who show warning signs can be classified as group B patients. Reference can be made to Handbook for Clinical Management of Dengue published by the World Health Organization (WHO) for relevant judgment principles.

The dengue fever patients with “severe symptoms” mentioned herein are those who show such signs as severe plasma leakage which causes shock and fluid accumulation with respiratory distress, severe bleeding, and severe organ impairment. In other embodiments, the severe patients can be classified as group C patients. Reference can be made to Handbook for Clinical Management of Dengue published by the WHO for relevant judgment principles.

The “severe dengue infection group” mentioned herein are determined according to the grouping method of the present invention. Generally speaking, in the medical detection field, α represents false positive or is referred to as the opposite of specificity; and β represents false negative or is referred to as the opposite of sensitivity. In application of the grouping method of the present invention, a non-structural protein 1 (NS1) and an endogenous anti-NS1 antibody of dengue virus in an ex vivo biological specimen are detected and crossly compared, and the subject for which at least one of the first test result and the second test result is positive is classified as the severe dengue infection group, thus effectively reducing the numerical value of β (namely, reducing “the false negative rate”) and improving the grouping accuracy rate (also referred to as “grouping accuracy”) of patients with severe dengue infection (namely, increasing the numerical value of “1-β”). In addition, when both of the first test result and the second test result are negative, it is determined that the subject corresponding to the ex vivo biological specimen is classified as a non-severe dengue infection group.

In the aforementioned embodiments, the test step can include but be not limited to an ELISA, western blot analysis, lateral laminar flow immunoassay, multiple immunoassay, radio immunoassay, immunoradiometric analysis, fluorescence immunoassay, chemiluminescence immunoassay and/or immunoturbidimetry. In other embodiments, other means can also be used to detect the NS1 and the endogenous anti-NS1 antibody in the ex vivo biological specimen.

It should be additionally noted that, the conventional test processes mostly determine the severity of the dengue fever patients in order according to a single test result, failing to effectively reduce the false negative rate of the test results. In some specific examples, compared to the false negative rate of about 10% to 30% in the current test process, the grouping method for “the severe dengue infection group” of the present invention can decrease the false negative rate of the test results to about 4.5% after diagnosis and confirmation by clinicians.

Thereinafter, it will be understood that particular configurations, aspects, examples, clauses and embodiments described hereinafter are shown by way of illustration and not as limitations of the invention. The principal features of this invention can be employed in various embodiments without departing from the scope of the invention. Thus, one skilled in the art can easily ascertain the essential characteristics of the present invention and, without departing from the spirit and scope thereof, can make various changes and modifications of the invention to adapt it to various usages and conditions.

### EXAMPLE 1

### 1. Virus Strain

A dengue virus serotype 1 (DENV 1, Taiwan virus strain 8700828), serotype 2 (DENV 2, virus strain 16681 and Taiwan virus strain 454009 A), serotype 3 (DENV 3, Taiwan virus strain 8700829), and serotype 4 (DENV 4, Taiwan virus strain 59201818) could be replicated in C6/36 cells using a conventional culture method. Virus culture was known to those of ordinary skill in the art of the present invention, so the details were not described herein. By using a commercially available centrifugation apparatus (for example, Macrosep® Advance Centrifugal Devices with a molecular weight cut-off of 30 kDa, Pall Corp., Port Washington, N.Y.), the supernatant after removal of cells was concentrated into DENV with a high viral titer at a rotation speed of 6000×g at 4° C., and then the DENV was stored in an environment lower than −70° C. for later use.

### 2. Serum Collection

This example used sera from 67 confirmed dengue patients, obtained in an acute phase (0-7 days after the onset of the disease) of these patients by the National Cheng Kung University Hospital (NCKUH) during the DENV outbreak in Tainan, Taiwan in 2015. The above sera were detected for dengue virus infection according to the laboratory standards established by the Taiwan Department of Disease Control. According to the latest principles published by the WHO, the dengue fever patients could be classified into severe patients, mild patients who had warning signs, and mild patients who had no warning signs according to the severity of the disease. In addition, this example used sera of 26 healthy volunteers as a negative control group. The collection of all the sera was carried out in accordance with the relevant criteria and regulations (IRB # A-BR-101-140) approved by the Institutional Review Board (IRB) of the NCKUH, and the informed consent of all participants and/or their legal representatives was obtained.

### 3. Test of NS1 and/or NS1 Complex

The test of the NS1 and/or NS1 complex in the sera of the subjects could be conducted in a conventional test manner or a manner disclosed in the patent with Taiwan patent publication No. I624668, which both were incorporated herein by reference.

This example used a commercially available set (for example, SD BIOLINE™ Dengue Duo set, Standard diagnostic Inc.) according to the manufacturer's operation manual. Briefly, an observation window of a rapid test displayed a control line (marked by C) and a test line (marked by T), where the test line contained 1 μL mouse anti-NS1 monoclonal antibody (model No. 12100/12110, Leadgene Biomedical Co., Inc., Taiwan) as a capture antibody. A plastic gold pad of the rapid test contained a mouse anti-NS1 monoclonal antibody—colloidal gold, as well as a 1 μL sheep anti-thrombin polyclonal antibody as a detection antibody. Then, 80 μL of a serum sample was added to a sample pad of the rapid test for reaction. At 15 minutes of the reaction, the result read with the naked eyes was equivalent to a first test result.

### 4. ELISA

For indirect ELISA, 50 μl NS1, bovine serum albumin (BSA), and peptides-conjugated BSA or an antibody (2 μg/ml, dissolved in PBS with pH 7.3) were separately coated in a 96-well ELISA culture plate, and the plate was then placed at 4° C. overnight. After blocking with PBS containing 1% BSA for 1 hour, the antibody [namely, the anti-NS1-WD peptide hAb, the hAb against NS1 of all serotypes (recognizing the 114^{th} to 119^{th} amino acids of the NS1), the anti-NS1-WD peptide mouse antibody (a monoclonal antibody strain 33D2; refer to SCIENTIFIC REPORTs 7: 6975, DOI:10.1038/s41598-017-07308-3, which was incorporated herein by reference), or the mouse antibody against NS1 of all serotypes (a monoclonal antibody strain 19-5, recognizing the 114^{th} to 119^{th} amino acid residues of the NS1, with; refer to SCIENTIFIC REPORTs 7: 6975, which was incorporated herein by reference)] or the patient serum (diluted at 1:50) was cultured in the wells and the reaction lasted for 1 hour at 37° C. The heavy chain variable regions CDRs and the light chain variable regions CDRs of the anti-NS1-WD peptide hAb and the hAb against NS1 of all serotypes were identical to those of the corresponding mouse monoclonal antibody strain above, but the remaining sequences were replaced with the sequences of the hAb. Results of the test using the anti-NS1-WD peptide mouse antibody and the mouse antibody against NS1 of all serotypes served as the comparative example. The sequences of the hAb other than the CDR sequences were known to those of ordinary skill in the art of the present invention, so the details were not described herein. Afterwards, the anti-human IgG (Jackson ImmunoResearch Laboratories, West Grove, Pa.) secondary antibody (diluted at 1:10000) of conjugated horseradish peroxidase (HRP) was added to the wells, and the reaction lasted for another 1 hour at 37° C. After the wells were washed with PBST, tetramethylbenzidine (TMB; Clinical Science Products, Mansfield, Mass.) was used as the substrate for chromogenic reaction. Subsequently, a termination solution (2N H₂SO₄) was added to the wells to stop reaction, and a commercially available microplate reader (for example, the VersaMax microplate reader; Molecular Devices, Sunnyvale, Calif.) was used to read the absorbance at OD450nm, obtaining a result, equivalent to a second test result.

### 5. Statistical Analysis

All numerical values were analyzed with Prism software (GraphPad, San Diego, Calif.). All the results were analyzed by means of unpaired Student's t-test or one-way ANOVA, so as to compare two or more independent groups. All the numerical values were obtained from three independent tests and expressed as mean±SD. In the statistical significance settings, the symbol * represented p<0.05, the symbol ** represented p<0.01, and the symbol *** represented p<0.001; and ns represented no significant difference in a 95% two-tailed confidence interval.

### EXAMPLE 2

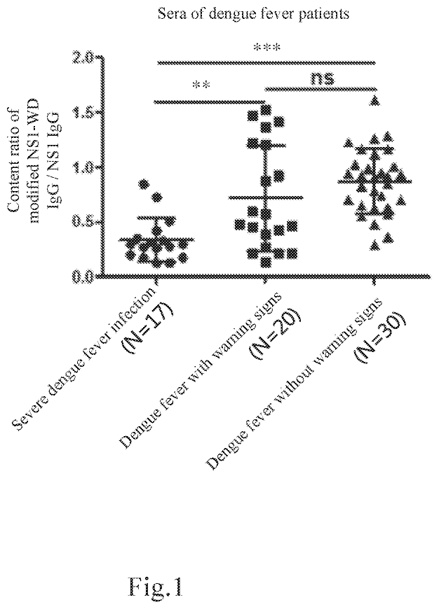

Following the method of example 1, an optical density (OD) value of the antibody (IgG) against modified NS1-WD peptide and an OD value of the antibody (IgG) against NS1 of all serotypes in the sera of the patients were separately detected, and then a ratio (NS1-WD IgG/NS1 IgG) of the OD values was used to evaluate the dengue fever patients varying in severity. The result was shown in FIG. 1.

Referring to FIG. 1, FIG. 1 showed a content ratio of anti-modified NS1-WD IgG/anti-NS1 IgG in sera of various dengue fever patients according to an example of the present invention, where the symbol * represented p<0.05, the symbol ** represented p<0.01, and the symbol *** represented p<0.001.

As shown in FIG. 1, compared to the sera (N=20) of the dengue fever patients having warning signs or the sera (N=30) of the dengue fever patients having no warning signs, the sera (N=17) of the patients with severe dengue infection had a significantly reduced content ratio of anti-NS1-WD IgG/anti-NS1 IgG. After further analysis, there was no statistically significant difference between the OD values of anti-NS1 IgG of all the patients, which indicated that the test of the anti-NS1-WD peptide antibody indeed facilitated improvement of the grouping accuracy of the dengue fever patients.

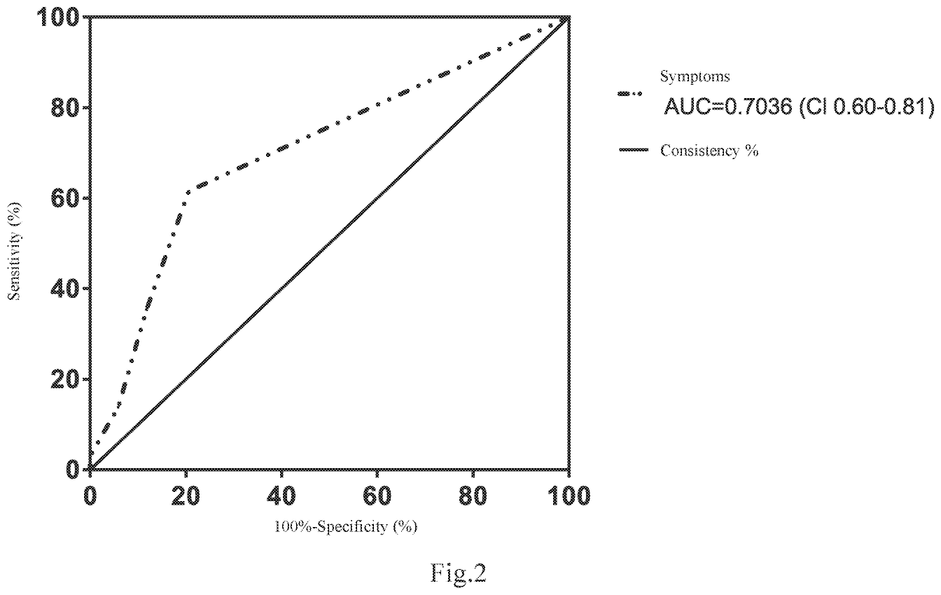

Referring to FIG. 2, FIG. 2 showed a consistency result for specificity and sensitivity of prediction of subjects with severe dengue infection according to a conventional method. The conventional method used in FIG. 2 included analyzing the consistency for specificity and sensitivity in terms of the symptoms, a medical history, and a simple non-specific test result (including a single test result or a test result without targeting anti-NS1-WD IgG) of the patients.

It could be seen from the result of FIG. 2 that the sensitivity of the conventional test method (only based on the symptoms, medical history, and simple non-specific test result) was higher than the specificity, where the area under curve (AUC) was 0.7036 (with a confidence interval of 0.60 to 0.81).

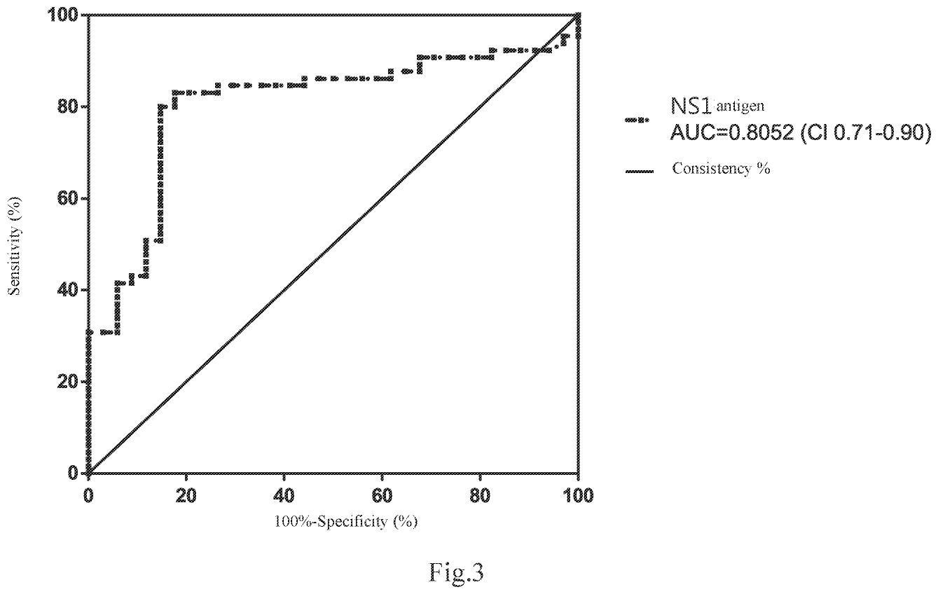

Referring to FIG. 3, FIG. 3 showed a consistency result for specificity and sensitivity of prediction of subjects with severe dengue infection in terms of NS1 antigens in the sera of dengue fever patients according to an example of the present invention. It could be seen from the result of FIG. 3 that the sensitivity of the test method in terms of the NS1 antigens in the sera of dengue fever patients was also higher than the specificity, where the AUC was 0.8052 (with a confidence interval of 0.71 to 0.90).

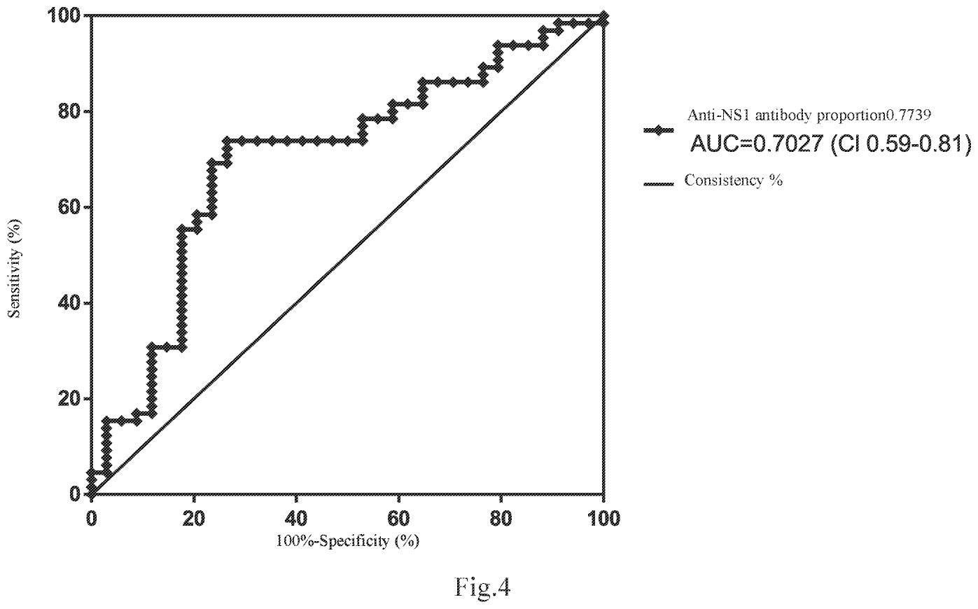

Referring to FIG. 4, FIG. 4 showed a consistency result (equivalent to a second test result) for specificity and sensitivity of prediction of subjects with severe dengue infection in terms of a proportion of the endogenous anti-NS1 antibody in the sera of dengue fever patients according to an example of the present invention. It could be seen from the result of FIG. 4 that the sensitivity of the test method in terms of the endogenous anti-NS1 antibody in the sera of the dengue fever patients was also higher than the specificity, where the AUC was 0.7027 (with a confidence interval of 0.59 to 0.81).

Referring to TABLE 1, TABLE 1 showed the number of positive and negative patients obtained by detecting and crossly comparing the NS1 antigen and the proportion of the endogenous anti-NS1 antibody in the sera of dengue fever patients according to an example of the present invention, and a result confirmed after differential diagnosis, where the severe and mild diseases were confirmed by post differential diagnosis for the subjects according to Handbook for Clinical Management of Dengue published by the WHO.

TABLE 1

| | Severe | Mild |
|---|---|---|
| 99 in total | (65) | (34) |
| NS1 antigen (+) or anti-NS1 antibody | 62 | 13 |
| proportion (+) | | |
| NS1 antigen (−) and anti-NS1 antibody | 3 | 21 |
| proportion (−) | | |

It could be seen from TABLE 1 that, the proportion of the endogenous anti-NS1 antibody was detected by using the hAb, and when at least one of the NS1 and the endogenous anti-NS1 antibody in the sera of the dengue fever patients was detected to be positive, it was determined that the subject corresponding to the ex vivo biological specimen was classified as the severe dengue infection group. After confirmation through post diagnosis or differential diagnosis, the grouping accuracy rate (also referred to as grouping accuracy) of predicting severe dengue infection by using the method of the present invention could reach up to 95.5% (namely, 62/65=95.5%), which was equivalent to that the false negative rate (namely, the value of β) was reduced to 4.5%. Therefore, the grouping method of the present invention was applicable to prediction of patients with severe dengue infection, and could be used as a reference for clinical staff to assess the risk and/or make treatment strategies.

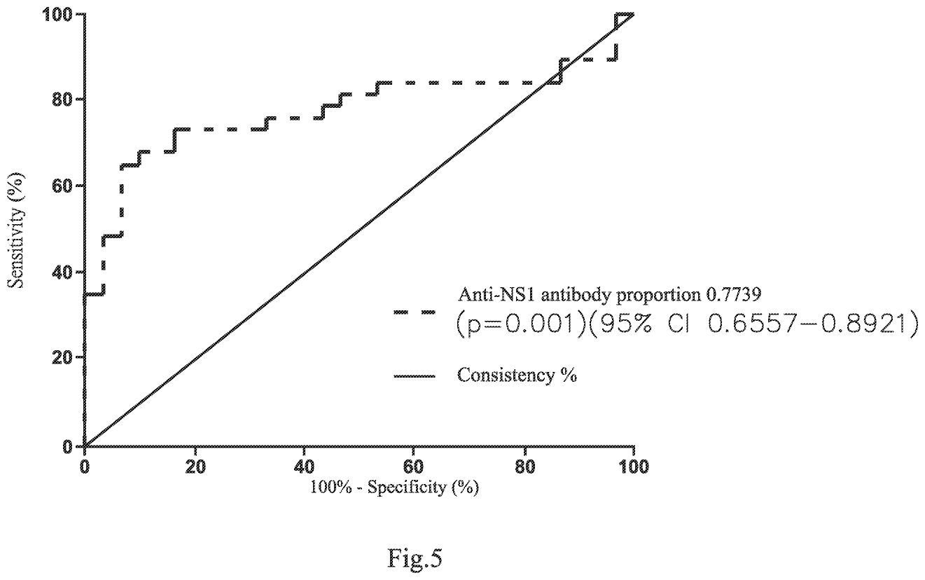

In comparison, if the dengue fever patients were grouped by detecting the proportion of the endogenous anti-NS1 antibody with a mouse antibody, the resulting false negative rate was high. Referring to FIG. 5, FIG. 5 showed a consistency result (equivalent to a second test result) for specificity and sensitivity of prediction of subjects with severe dengue infection in terms of a proportion of the endogenous anti-NS1 antibody in the sera of dengue fever patients by means of mouse antibody detection according to a comparative example. It could be seen from the result of FIG. 5 that the sensitivity (72.97%) of the test method in terms of the endogenous anti-NS1 antibody in the sera of dengue fever patients with the mouse antibody was lower than the specificity (83.33%), where the AUC was 0.7739 (with a confidence interval of 0.6557 to 0.8921).

Referring to TABLE 2, TABLE 2 showed the number of positive and negative patients obtained by detecting the proportion of the endogenous anti-NS1 antibody in the sera of dengue fever patients with the mouse antibody according to the comparative example, and a result confirmed after differential diagnosis, where the severe and mild diseases were confirmed by post differential diagnosis for the subjects according to Handbook for Clinical Management of Dengue published by the WHO.

TABLE 2

| | Severe | Mild |
|---|---|---|
| 67 in total | (37) | (30) |
| Anti-NS1 antibody proportion (+) | 27 | 5 |
| Anti-NS1 antibody proportion (−) | 10 | 25 |

It could be seen from TABLE 2 that, when the endogenous anti-NS1 antibody in the sera of the dengue fever patients was detected to be positive with the mouse antibody, it was determined that the subject corresponding to the ex vivo biological specimen was classified as the severe dengue infection group. After confirmation through post diagnosis or differential diagnosis, the grouping accuracy rate (also referred to as grouping accuracy) of predicting subjects with severe dengue infection by the method in the comparative example was merely 72.9% (namely, 27/37=72.9%), and the false negative rate reached up to 27.1% (namely, 10/37=27.1%), which was indeed much higher than the false negative rate (4.5%) of the grouping method of the present invention.

To sum up, the above-described specific antigen, specific antibody, specific patient group, specific analysis mode, or specific evaluation method is merely used to illustrate the method of elevating prediction accuracy of grouping subjects with severe dengue infection. However, those of ordinary skill in the art of the present invention should understand that other antigens, other antibodies, other patient groups, other analysis modes, or other evaluation methods can also be used in the method of elevating prediction accuracy of grouping patients with severe dengue infection without departing from the spirit and scope of the present invention, so the present invention is not limited to the above description. For example, without affecting the grouping accuracy, the first test result can be obtained by detecting the NS1 and/or NS1 complex in other manners or the second test result can be obtained by detecting the endogenous anti-NS1 antibody in other manners.

It can be seen from the aforementioned embodiments that, in the method of elevating prediction accuracy of grouping patients with severe dengue infection in the present invention, a non-structural protein 1 (NS1) and an endogenous anti-NS1 antibody of dengue virus in an ex vivo biological specimen can be detected and crossly compared, leading in reduction of false negative rates of testing results, as well as elevating prediction accuracy of grouping patients with severe dengue infection.

Although the present invention has been disclosed above with several specific examples, various modifications, changes and substitutions can be made to the foregoing disclosure. Moreover, it should be understood that, without departing from the spirit and scope of the present invention, certain features of the examples of the present invention will be used in some cases, but other features are not used correspondingly. Therefore, the spirit and the scope of the claims of the present invention should not be limited to those described in the above exemplary examples.

What is claimed is:

1. A method of improving prediction accuracy of severe dengue infection, comprising:
providing an ex vivo biological specimen, wherein the ex vivo biological specimen has not been diagnosed or differentially diagnosed with a dengue virus infection or a suspected dengue virus infection;
performing at least one detection step on the ex vivo biological specimen, for obtaining a first test result and a second test result, wherein the first test result is corresponding to a non-structural protein 1 (NS1) and/or an NS1 complex of dengue virus, and the second test result is corresponding to an endogenous anti-NS1 antibody; and
crossly comparing the first test result and the second test result, for obtaining a grouping result, wherein when at least one of the first test result and the second test result is positive, a subject corresponding to the ex vivo biological specimen is classified as a severe dengue infection group.

2. The method of claim 1, wherein the ex vivo biological specimen comprises blood, urine, saliva, tissue fluid and/or lymphatic fluid.

3. The method of claim 1, wherein the first test result is obtained by detecting the NS1 and/or NS1 complex with an antibody the antibody is an exogenous anti-NS1 antibody, and the NS1 complex comprises the NS1-thrombin or NS1-prothrombin.

4. The method of claim 1, wherein a serotype of the dengue virus comprises type 1, type 2, type 3 and type 4.

5. The method of claim 1, wherein a humanized antibody (hAb) specifically recognizes the endogenous anti-NS1 antibody, and the endogenous anti-NS1 antibody comprises a first antibody and a second antibody, the first antibody specifically recognizes the 109^{th} to the 122^{nd} amino acid residues of the NS1, and the second antibody specifically recognizes the 114^{th} to the 119^{th} amino acid residues.

6. The method of claim 5, wherein an isotype of the endogenous anti-NS1 antibody is IgG and/or IgM, and the second test result is represented by a content ratio of the first antibody to the second antibody.

7. The method of claim 1, wherein the subject is a mammal.

8. The method of claim 1, wherein the subject is a human being.

9. The method of claim 1, wherein both of the first test result and the second test result are negative, the subject corresponding to the ex vivo biological specimen is classified as a non-severe dengue infection group.

10. The method of claim 1, wherein the at least detection step comprises enzyme-linked immunosorbent assay (ELISA), western blotting assay, lateral flow immunoassay, multiple immunoassay, radioimmunoassay, immunoradiometric assay (IRMA), fluorescence immunoassay (FIA), chemilluminescence immunoassay (CLIA) and/or immunoturbidimetry.

11. A method of improving prediction accuracy of severe dengue infection, comprising:
providing an ex vivo biological specimen, wherein the ex vivo biological specimen has not been diagnosed or differentially diagnosed with a dengue virus infection or a suspected dengue virus infection;
performing at least one detection step on the ex vivo biological specimen, for obtaining a first test result and a second test result, wherein the first test result is corresponding to a non-structural protein 1 (NS1) and/or an NS1 complex of dengue virus, and the second test result is corresponding to an endogenous anti-NS1 antibody; and
crossly comparing the first test result and the second test result, for obtaining a grouping result, wherein the second test result is represented by a content ratio of the first antibody to the second antibody, when at least one of the first test result and the second test result is positive, a subject corresponding to the ex vivo biological specimen is classified as a severe dengue infection group.

12. The method of claim 11, wherein the ex vivo biological specimen comprises blood, urine, saliva, tissue fluid and/or lymphatic fluid.

13. The method of claim 11, wherein the first test result is obtained by detecting the NS1 and/or NS1 complex with an antibody the antibody is an exogenous anti-NS1 antibody, and the NS1 complex comprises the NS1-thrombin or NS1-prothrombin.

14. The method of claim 11, wherein a serotype of the dengue virus comprises type 1, type 2, type 3 and type 4.

15. The method of claim 11, wherein a humanized antibody (hAb) specifically recognizes the endogenous anti-NS1 antibody, and the endogenous anti-NS1 antibody comprises a first antibody and a second antibody, the first antibody specifically recognizes the 109^{th} to the 122^{nd} amino acid residues of the NS1, and the second antibody specifically recognizes the 114^{th} to the 119^{th} amino acid residues.

16. The method of claim 15, wherein an isotype of the endogenous anti-NS1 antibody is IgG and/or IgM.

17. The method of claim 11, wherein the subject is a mammal.

18. The method of claim 11, wherein the subject is a human being.

19. The method of claim 11, wherein both of the first test result and the second test result are negative, the subject corresponding to the ex vivo biological specimen is classified as a non-severe dengue infection group.

20. The method of claim 11, wherein the at least detection step comprises enzyme-linked immunosorbent assay (ELISA), western blotting assay, lateral flow immunoassay, multiple immunoassay, radioimmunoassay, immunoradiometric assay (IRMA), fluorescence immunoassay (FIA), chemilluminescence immunoassay (CLIA) and/or immunoturbidimetry.
